(19) United States
(12) Patent Application Publication
(10) Pub. No.: US 2025/0123270 A1
(43) Pub. Date: Apr. 17, 2025

(54) METHODS FOR PREDICTING AUTOIMMUNITY POST GENE THERAPY

(71) Applicant: Children's Hospital Medical Center, Cincinnati, OH (US)

(72) Inventors: Akihiro ASAI, Mason, OH (US); Kokoro SAKABE, Cincinnati, OH (US); Eriko KISHIMOTO, Hyogo (JP)

(21) Appl. No.: 18/915,716

(22) Filed: Oct. 15, 2024

Related U.S. Application Data
(60) Provisional application No. 63/590,258, filed on Oct. 13, 2023.

(51) Int. Cl. G01N 33/50

(52) U.S. Cl. CPC G01N 33/5038

(57) ABSTRACT

A method for detecting the presence of an allo-antibody against bile salt export pump (BSEP) protein in a subject, comprising: (a) providing a cell culture vessel comprising (i) an upper chamber and a lower chamber, wherein the chambers comprise a culture medium for culturing hepatocytes, (ii) a permeable membrane separating the chambers, and (iii) a layer of hepatocyte-like cells grown on the permeable membrane that are differentiated from a population of pluripotent stem cells having a modified ABCB11 gene; (b) adding taurocholic acid (TCA) to the lower chamber; (c) adding a serum sample from the subject to the upper chamber; (d) collecting culture medium from each chamber; and (e) determining the concentration of TCA in the culture medium from each chamber; wherein the lack of TCA in the culture medium of the upper chamber indicates that the sample contains allo-antibody against BSEP.

### CROSS-REFERENCE TO RELATED APPLICATIONS

This application claims the benefit of the filing date of U.S. Provisional Application No. 63/590,258, filed Oct. 13, 2023, the entire contents of which are incorporated by reference herein.

### BACKGROUND OF THE INVENTION

Progressive familial intrahepatic cholestasis (PFIC) is a rare hereditary disorder in which the ability to drain bile from the liver though the bile ducts is impaired (cholestasis) owing to a deficiency in bile salt export pump (BSEP) protein and can lead to liver disease and liver failure. Since bile flow is dependent on efficient bile acid transport by hepatocytes, genetic defects affecting bile acid transporters, which disturb the canalicular export of bile acids and result in cholestasis. The characteristic pattern of clinical presentation includes jaundice, pruritus, elevated serum bile acid levels, fat malabsorption, fat soluble vitamin deficiency, and liver injury. PFIC2 is a specific deficiency caused by variants in the ABCB11 gene encoding BSEP protein expressed at the canalicular membrane. Liver transplant is the standard treatment for infants with severe manifestation of PFIC2.

Cholestasis often does not respond to medical therapy of any sort. Some reports indicate success in children with chronic cholestatic diseases with the use of ursodeoxycholic acid, which acts to increase bile formation and antagonizes the effect of hydrophobic bile acids on biological membranes. Phenobarbital may also be useful in some children with chronic cholestasis.

Bile acids, the major component of bile, are cholesterol metabolites that are formed in the liver and secreted into the duodenum of the intestine, where they have important roles in the solubilization and absorption of dietary lipids and vitamins. Most bile acids (^{˜}95%) are subsequently reabsorbed in the ileum and returned to the liver via the enterohepatic circulatory system. Hepato-enteric recirculation of bile acids regulates a balance between de novo synthesis and sinusoid-to-canalicular transport of bile acids in hepatocytes. This is mediated by the intracellular accumulation of bile acids. Since bile flow is dependent on efficient bile acid transport by hepatocytes, genetic defects affecting bile acid transporters, which disturb the canalicular export of bile acids and result in cholestasis. The characteristic pattern of clinical presentation includes jaundice, pruritus, elevated serum bile acid levels, fat malabsorption, fat soluble vitamin deficiency, and liver injury.

Cholestasis often does not respond to medical therapy of any sort. Some reports indicate success in children with chronic cholestatic diseases with the use of ursodeoxycholic acid, which acts to increase bile formation and antagonizes the effect of hydrophobic bile acids on biological membranes. Phenobarbital may also be useful in some children with chronic cholestasis.

Treatment of fat malabsorption principally involves dietary substitution. In older patients, a diet that is rich in carbohydrates and proteins can be substituted for a diet containing long-chain triglycerides. In infants, that may not be possible, and substitution of a formula containing medium-chain triglycerides may improve fat absorption and nutrition.

In chronic cholestasis, careful attention must be paid to prevent fat-soluble vitamin deficiencies, which are common complications in pediatric patients with chronic cholestasis. This is accomplished by administering fat-soluble vitamins and monitoring the response to therapy. Oral absorbable, fat-soluble vitamin formulation A, D, E, and K supplementation is safe and potentially effective in pediatric patients with cholestasis.

Other treatments include gene therapy and liver transplantation. However, these therapies have their own drawbacks and side effects that may be problematic. Autoimmune reaction to a gene therapy, including transplant, for genetic disorders is an emerging crucial problem. There currently is no experimental system to predict if a gene therapy will likely induce an autoimmune reaction in an individual receiving gene therapy, including, for example, receiving cell or organ transplants. It is well known that a subject having PFIC2 deficiency and receiving a liver transplant often develops allo-immune antibodies against BSEP, which cause an antibody-mediated graft rejection. This suggests that a gene therapy for such a PFIC2 patient induces an autoimmune response in the patient and the transplanted hepatocytes with a BSEP transgene are targeted by the host immune system and result in recurrence of BSEP deficiency.

Therefore, there is a need to develop a system to predict if a gene therapy or transplantation will likely induce an autoimmune reaction in an individual receiving gene therapy or transplanted tissue.

### SUMMARY OF THE INVENTION

The present invention is directed to methods for predicting autoimmunity post gene therapy, and, more specifically, to methods for determining the presence in a sample from a subject of an allo-immune antibody against bile salt export pump (BSEP) protein. The methods employ a hepatocyte culture system which employs hepatocyte-like cells obtained from a population of pluripotent stem cells from the subject. The presence of such allo-immune antibodies is predictive of the subject developing an autoimmune reaction to gene therapy.

The methods employ a population of hepatocyte-like cells. In a specific embodiment, the hepatocyte-like cells are obtained by a method comprising (i) culturing a population of pluripotent stem cells in an endoderm differentiation medium, wherein the pluripotent stem cells comprise a genetically modified ABCB11 gene; (ii) culturing a population of cells obtained from step (i) in a hepatic specification medium; and (iii) culturing a population of cells obtained from step (ii) in a hepatocyte maturation medium to produce a population of hepatocyte-like cells. In a further embodiment, the genetically modified ABCB11 gene expresses a truncated mutant of a BSEP protein. In a specific embodiment, the truncated mutant of the BSEP protein is a R1090X truncation mutant and, more specifically, the genetic modification of the ABCB11 gene is performed by CRISPR/Cas9-mediated gene editing. In a further embodiment, the pluripotent stem cells are induced pluripotent stem cells (iPSCs).

A new method of detecting allo-antibodies in a sample utilizes this culture system. The method for detecting the presence of an allo-antibody against bile salt export pump (BSEP) protein in a subject, comprises:
(a) providing a cell culture vessel comprising (i) an upper chamber and a lower chamber, wherein both the upper chamber and the lower chamber comprise a culture medium for culturing hepatocytes, (ii) a permeable membrane separating the upper chamber and the lower chamber, and (iii) a layer of hepatocyte-like cells grown on the permeable membrane, wherein the hepatocyte-like cells are differentiated from a population of pluripotent stem cells having a modified ABCB11 gene;
(b) adding taurocholic acid (TCA) to the lower chamber culture medium;
(c) adding a serum sample from the subject to the upper chamber culture medium;
(d) after a period of time, collecting culture medium from each of the upper chamber and the lower chamber; and
(e) determining the concentration of TCA in the culture medium from each of the upper chamber and the lower chamber;
   wherein the lack of TCA in the culture medium of the upper chamber indicates that the sample contains allo-antibody against BSEP.

The details of this method, each component, and the steps of the method, along with other embodiments, are discussed in greater detail below.

### BRIEF DESCRIPTION OF DRAWINGS

FIG. 1 shows attributes of the progressive familial intrahepatic cholestasis (PFIC) and problems associated with PFIC.

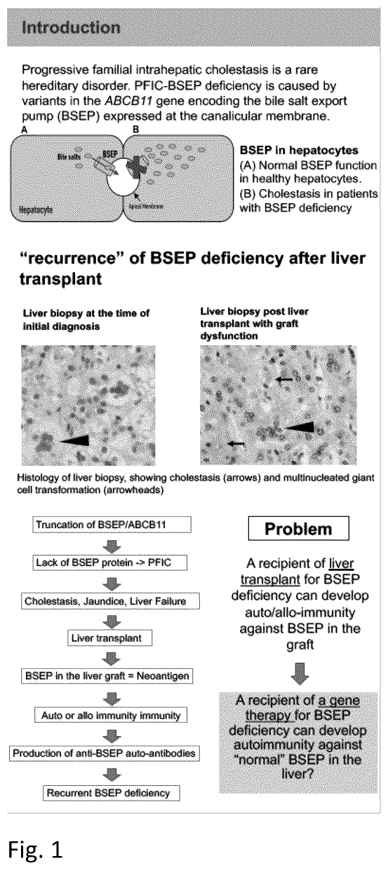

FIG. 2 shows a method of studying complications after gene therapy for PFIC in vitro.

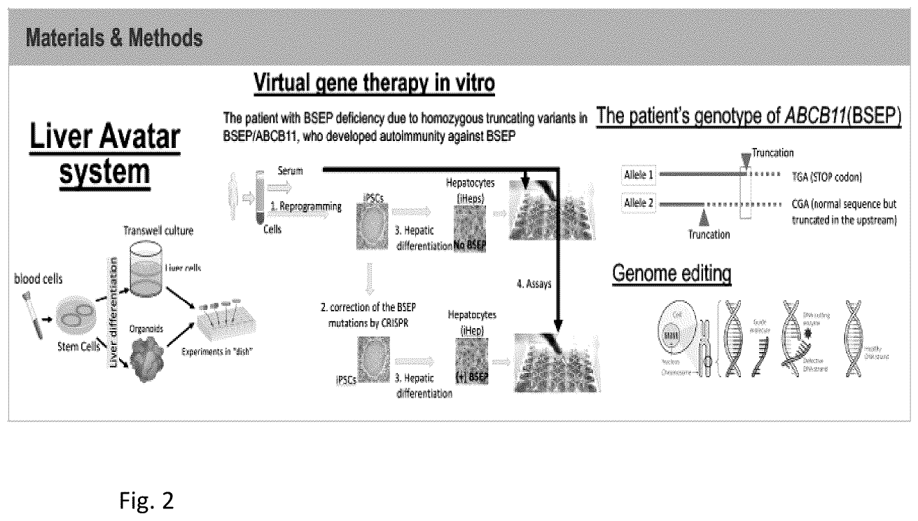

FIG. 3 shows the results of the disclosed method for detecting allo-antibodies in a sample utilizes this culture system.

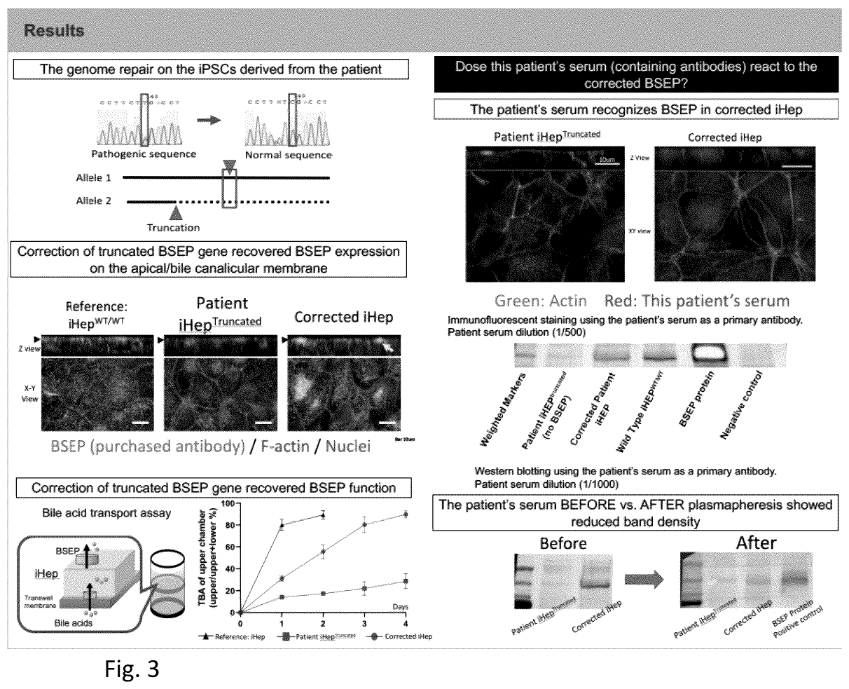

### DETAILED DESCRIPTION OF THE INVENTION

Specific embodiments of the invention are described herein. The invention can, however, be embodied in different forms and should not be construed as limited to the embodiments set forth herein. Rather, these embodiments are provided to illustrate more specific features of certain embodiments of the invention to those skilled in the art.

The terminology as set forth herein is for description of the embodiments only and should not be construed as limiting the disclosure as a whole. All references to singular characteristics or limitations of the present disclosure shall include the corresponding plural characteristic or limitation, and vice versa, unless otherwise specified or clearly implied to the contrary by the context in which the reference is made. Unless otherwise specified, “a,” “an,” “the,” and “at least one” are used interchangeably. Furthermore, as used in the description and the appended claims, the singular forms “a,” “an,” and “the” are inclusive of their plural forms, unless the context clearly indicates otherwise.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to +20%, preferably up to +10%, more preferably up to +5%, and more preferably still up to +1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

To the extent that the term “includes” or “including” is used in the description or the claims, it is intended to be inclusive of additional elements or steps, in a manner similar to the term “comprising” as that term is interpreted when employed as a transitional word in a claim. Furthermore, to the extent that the term “or” is employed (e.g., A or B), it is intended to mean “A or B or both.” When the “only A or B but not both” is intended, then the term “only A or B but not both” is employed. Thus, use of the term “or” herein is the inclusive, and not the exclusive use. When the term “and” as well as “or” are used together, as in “A and/or B” this indicates A or B as well as A and B.

All ranges and parameters, including but not limited to percentages, concentrations, temperatures, parts, ratios and other numerical parameters disclosed herein are understood to encompass any and all sub-ranges subsumed therein, and every number between the endpoints. For example, a stated range of “1 to 10” should be considered to include any and all sub-ranges beginning with a minimum value of 1 or more and ending with a maximum value of 10 or less (e.g., 1 to 6.1, or 2.3 to 9.4), and to each integer (1, 2, 3, 4, 5, 6, 7, 8, 9, and 10) contained within the range.

Any combination of method or process steps as used herein can be performed in any order, unless otherwise specified or clearly implied to the contrary by the context in which the referenced combination is made.

The present invention is directed to a method for predicting autoimmunity post gene therapy, and, more specifically, to methods for determining the presence in a sample from a subject of an allo-immune antibody against bile salt export pump (BSEP) protein.

The methods employ a population of hepatocyte-like cells. In a specific embodiment, the hepatocyte-like cells are obtained by a method comprising (i) culturing a population of pluripotent stem cells in an endoderm differentiation medium, wherein the pluripotent stem cells comprise a genetically modified ABCB11 gene; (ii) culturing a population of cells obtained from step (i) in a hepatic specification medium; and (iii) culturing a population of cells obtained from step (ii) in a hepatocyte maturation medium to produce a population of hepatocyte-like cells. In a further embodiment, the genetically modified ABCB11 gene expresses a truncated mutant of a BSEP protein. In a specific embodiment, the truncated mutant of the BSEP protein is a R1090X truncation mutant and, more specifically, the genetic modification of the ABCB11 gene is performed by CRISPR/Cas9-mediated gene editing. In a further embodiment, the pluripotent stem cells are induced pluripotent stem cells (iPSCs). Such methods are disclosed, for example, in WO 2020/097555 A1, the entirety of which is incorporated herein by reference.

In one embodiment, a method for detecting the presence of an allo-antibody against bile salt export pump (BSEP) protein in a subject, comprises
(a) providing a cell culture vessel comprising (i) an upper chamber and a lower chamber, wherein both the upper chamber and the lower chamber comprise a culture medium for culturing hepatocytes, (ii) a permeable membrane separating the upper chamber and the lower chamber, and (iii) a layer of hepatocyte-like cells grown on the permeable membrane, wherein the hepatocyte-like cells are differentiated from a population of pluripotent stem cells having a modified ABCB11 gene;
(b) adding taurocholic acid (TCA) to the lower chamber culture medium;
(c) adding a serum sample from the subject to the upper chamber culture medium;
(d) after a period of time, collecting culture medium from each of the upper chamber and the lower chamber; and
(e) determining the concentration of TCA in the culture medium from each of the upper chamber and the lower chamber;
   wherein the lack of TCA in the culture medium of the upper chamber indicates that the sample contains allo-antibody against BSEP.

In another embodiment, a method of predicting autoimmunity post gene therapy for PFIC2 comprises (a) providing a cell culture vessel comprising (i) an upper chamber and a lower chamber, wherein both the upper chamber and the lower chamber comprise a culture medium for culturing hepatocytes, (ii) a permeable membrane separating the upper chamber and the lower chamber, and (iii) a layer of hepatocyte-like cells grown on the permeable membrane, wherein the hepatocyte-like cells are differentiated from a population of pluripotent stem cells having a modified ABCB11 gene; (b) adding taurocholic acid (TCA) to the lower chamber culture medium; (c) adding a serum sample from the subject to the upper chamber culture medium; (d) after a period of time, collecting culture medium from each of the upper chamber and the lower chamber; and (e) determining the concentration of TCA in the culture medium from each of the upper chamber and the lower chamber; wherein the lack of TCA in the culture medium of the upper chamber indicates that the subject providing the sample will exhibit autoimmunity post gene therapy.

The present disclosure is based, at least in part, in the development of an in vitro disease model for BSEP deficiency, which can be used to improve understanding of genetic cholestatic liver disease and whether a particular gene therapy treatment is a viable option for a subject. The cell culture step of step (a) is a model that has been previously described in U.S. application Ser. No. 17/292,162, which was published as US 2021-0395679, entitled “IN VITRO CELL CULTURE SYSTEM FOR PRODUCING HEPATOCYTE-LIKE CELLS AND USES THEREOF”, the entirety of which is incorporated by reference herein.

The inventors have now taken that model further by incorporating the cell culture system into a method of detecting an allo-antibody in a sample. That is, the inventors have discovered that the same two chamber system can be further used to detect the presence or absence of an allo-antibody in a serum sample from a subject with liver disease. This detection method allows a medical professional to determine whether the subject is a candidate for gene therapy or a liver transplant. Further, the medical profession will be able to use this detection method to monitor the patient after gene therapy or a transplant to understand how the subject is reacting to the therapy. The culture method steps and component are described herein as well as the additional steps taken to utilize the culture system as a detection system.

### Methods of Producing Hepatocyte-Like Cells In Vitro

Aspects described herein stem from, at least in part, development of methods that efficiently direct differentiation of pluripotent stem (PS) cells into hepatocyte-like cells. In particular, the present disclosure provides, inter alia, an in vitro culturing process for producing a population of hepatocyte-like cells from pluripotent stem cells and the resultant hepatocyte-like cells show a functional apico-basolateral polarity, including canalicular function, specifically in bile acid transport and bile acid de novo synthesis, from unmodified pluripotent stem cells (e.g., from a human subject). In some embodiments, this culturing process may involve multiple differentiation stages (e.g., 2, 3, or more). Alternatively, or in addition, the culturing process may involve culture of the cells on a permeable membrane which separates and upper and lower chamber in a cell culture vessel. In some embodiment, the total time period for the in vitro culturing process described herein can range from about 17-27 days (e.g., 20-26 days, 20-23 days, or 19-23 days). In one example, the total time period is about 22 days.

In some embodiments, the methods for producing hepatocyte-like cells as disclosed herein may include multiple differentiation stages (e.g., 2, 3, 4, or more). For example, a endoderm differentiation step, e.g., the culturing of the hPS cells under differentiation conditions to obtain cells of the definitive endoderm (DE cells), a hepatic specification step, e.g., the culturing of the obtained DE cells under differentiation conditions to obtain the hepatic progenitor cells, and a hepatic maturation step, e.g., culturing the hepatic progenitor cells under conditions to obtain hepatocyte-like cells.

Existing methods for producing human hepatocytes often fail to form functional apico-basolateral polarity. Thus, there is a lack of a suitable experimental system for dynamic tracing of transcellular transport of bile acids The in vitro model described herein can provide a reliable source of hepatocyte-like cells with transcellular transport and de novo synthesis of bile acids. The pluripotent stem (PS) cell-derived hepatocyte-like cells can be used in various applications, including, e.g., but not limited to, as an in vitro model for modeling genetic cholestatic liver diseases or disorders, drug discovery and/or developments.

Accordingly, embodiments of various aspects described herein relate to methods for generation of hepatocyte-like cells from PS cells, cells produced by the same, and methods of use in a detection method to detect allo-antibodies in a serum sample.

### Pluripotent Stem Cells

In some embodiments, the in vitro culturing system disclosed herein may use pluripotent stem cells (e.g., human pluripotent stem cells) as the starting material for producing hepatocyte-like cells. As used herein, “pluripotent” or “pluripotency” refers to the potential to form all types of specialized cells of the three germ layers (endoderm, mesoderm, and ectoderm); and is to be distinguished from “totipotent” or “totipotency”, that is the ability to form a complete embryo capable of giving rise to offsprings. As used herein, “human pluripotent stem cells” (hPSC) refers to human cells that have the capacity, under appropriate conditions, to self-renew as well as the ability to form any type of specialized cells of the three germ layers (endoderm, mesoderm, and ectoderm). hPS cells may have the ability to form a teratoma in 8-12 week old SCID mice and/or the ability to form identifiable cells of all three germ layers in tissue culture. Included in the definition of human pluripotent stem cells are embryonic cells of various types including human embryonic stem (hES) cells, (see, e.g., Thomson et al. (1998), Heins et. al. (2004), as well as induced pluripotent stem cells [see, e.g. Takahashi et al., (2007); Zhou et al. (2009); Yu and Thomson in Essentials of Stem Cell Biology (2nd Edition]. The various methods described herein may utilize hPS cells from a variety of sources. For example, hPS cells suitable for use may have been obtained from developing embryos by use of a nondestructive technique such as by employing the single blastomere removal technique described in e.g. Chung et al (2008), further described by Mercader et al. in Essential Stem Cell Methods (First Edition, 2009). Additionally or alternatively, suitable hPS cells may be obtained from established cell lines or may be adult stem cells.

In some aspects, the pluripotent stem cells for use according to the disclosure may be human embryonic stem cells (hESs). Various techniques for obtaining hES cells are known to those skilled in the art. In some instances, the hES cells for use according to the present disclosure are ones, which have been derived (or obtained) without destruction of the human embryo, such as by employing the single blastomere removal technique known in the art. See, e.g., Chung et al., Cell Stem Cell, 2 (2): 113-117 (2008), Mercader et al., Essential Stem Cell Methods (First Edition, 2009). Suitable hES cell lines can also be used in the methods disclosed herein. Examples include, but are not limited to, cell lines SA167, SA181, SA461 (Cellartis AB, Goteborg, Sweden) which are listed in the NIH stem cell registry, the UK Stem Cell bank and the European hESC registry and are available on request. Other suitable cell lines for use include those established by Klimanskaya et al., Nature 444:481-485 (2006), such as cell lines MA01 and MA09, and Chung et al., Cell Stem Cell, 2 (2): 113-117 (2008), such as cell lines MA126, MA127, MA128 and MA129, which all are listed with the International Stem Cell Registry (assigned to Advanced Cell Technology, Inc. Worcester, MA, USA).

Alternatively, the pluripotent stem cells for use in the methods disclosed herein may be induced pluripotent stem cells (iPSCs) such as human iPSCs. As used herein “hiPS cells” refers to human induced pluripotent stem cells. hiPS cells are a type of pluripotent stem cells derived from non-pluripotent cells—typically adult somatic cells—by induction of the expression of genes associated with pluripotency, such as SSEA-3, SSEA-4, TRA-1-60, TRA-1-81, Oct-4, Sox2, Nanog and Lin28. Various techniques for obtaining such iPSC cells have been established and all can be used in the present disclosure. See, e.g., Takahashi et al., Cell 131(5):861-872 (2007); Zhou et al., Cell Stem Cell. 4(5):381-384 (2009); Yu and Thomson in Essentials of Stem Cell Biology (2nd Edition, Chapter 4)]. It is also envisaged that the endodermal and/or hepatic progenitor cells may also be derived from other pluripotent stem cells such as adult stem cells, cancer stem cells or from other embryonic, fetal, juvenile or adult sources.

### Genetic Modification of Pluripotent Stem Cells

In some embodiments, the pluripotent stem cells used in the in vitro culturing system disclosed herein for producing hepatocyte-like cells may be genetically modified such that the ABCB11 gene, which encodes a Bile Salt Export Pump (BSEP) protein, is disrupted. As used herein, the term “BSEP” is intended to mean the bile transporter bile salt export pump. Accordingly, the present disclosure also provides methods of preparing such genetically modified pluripotent stem cells. As used herein, the term “a disrupted gene” refers to a gene containing one or more mutations (e.g., insertion, deletion, or nucleotide substitution, etc.) relative to the wild-type counterpart so as to substantially reduce or completely eliminate the activity of the encoded gene product. The one or more mutations may be located in a non-coding region, for example, a promoter region, a regulatory region that regulates transcription or translation; or an intron region. Alternatively, the one or more mutations may be located in a coding region (e.g., in an exon). In some instances, the disrupted gene does not express or express a substantially reduced level of the encoded protein. In other instances, the disrupted gene expresses the encoded protein in a mutated form, which is either not functional or has substantially reduced activity. In some embodiments, a disrupted gene does not express (e.g., encode) a functional protein.

The ABCB11/BSEP protein contains 12 transmembrane domains and 2 intracellular nucleotide-binding domains. In some embodiments, the targeted modification of ABCB11/BSEP is at the R1090 position, located in exon 25. In a specific example, the modification results in a truncation at R1090 which induces a BSEP protein without a functional C-terminal domain, lacking the second nucleotide-binding domain of Walker A and B and a conserved signature C motif of ATP-binding cassette (ABC). The resulting peptide is a short BSEP with an unpaired, single, intracellular ABC domain. The instant disclosure demonstrates that truncated versions of BSEP, such as the R1090X mutant, exhibits dysfunction in hepatocyte-like cells.

In another exemplary embodiment, the targeted modification results in a truncating mutation, R1057X. The R1057X truncating mutation was studied in a transfection model in MDCK II cells and showed stable expression level but low transport activity. Kagawa et al., American Journal of Physiology Gastrointestinal and Liver Physiology 294:G58-6 (2008).

Alternatively, the genetically modified pluripotent stem cells may have a disrupted gene involved in a bile acid transport or synthesis pathway in hepatocytes, for example, a gene know or thought to be involved in a genetic cholestatic liver disease (e.g., Progressive Familial Intrahepatic Cholestasis (PFIC), Benign Recurrent Intrahepatic Cholestasis (BRIC), and Intrahepatic Cholestasis of Pregnancy (ICP)). Non-limiting examples of gene contributors of PFIC, BRIC, and/or ICP include ATP8B1/FIC1 (gene on chromosome 18q21-22), and ABCB4/MDR3 (gene on chromosome 7q21). As used herein, the term “MDR” is intended to mean multi-drug resistance transporter. MDR 1 and 3 are members of the ATP-binding cassette (ABC) family of transporters. MDR 1 is important in regulating the traffic of drugs, peptides and xenobiotics into the body and in protecting the body against xenobiotic insults and drug toxicity, while MDR 3 is essential for phospholipid secretion into bile.

Techniques such as CRISPR (particularly using Cas9 and guide RNA), editing with zinc finger nucleases (ZFNs) and transcription activator-like effector nucleases (TALENs) may be used to produce the genetically engineered pluripotent stem cells.

‘Genetic modification’, ‘genome editing’, or ‘genomic editing’, or ‘genetic editing’, as used interchangeably herein, is a type of genetic engineering in which DNA is inserted, deleted, and/or replaced in the genome of a targeted cell. Targeted genome modification (interchangeable with “targeted genomic editing” or “targeted genetic editing”) enables insertion, deletion, and/or substitution at pre-selected sites in the genome. When an endogenous sequence is deleted at the insertion site during targeted editing, an endogenous gene comprising the affected sequence may be knocked-out or knocked-down due to the sequence deletion. In another aspect, an endogenous gene may be modified by introducing a change in an endogenous gene codon, wherein the modification introduces an amino acid change in the gene product or introduction of a stop codon. Therefore, targeted modification may also be used to disrupt endogenous gene expression with precision. Similarly used herein is the term “targeted integration,” referring to a process involving insertion of one or more exogenous sequences, with or without deletion of an endogenous sequence at the insertion site. In comparison, randomly integrated genes are subject to position effects and silencing, making their expression unreliable and unpredictable. For example, centromeres and sub-telomeric regions are particularly prone to transgene silencing. Reciprocally, newly integrated genes may affect the surrounding endogenous genes and chromatin, potentially altering cell behavior or favoring cellular transformation. Therefore, inserting exogenous DNA in a pre-selected locus such as a safe harbor locus, or genomic safe harbor (GSH) is important for safety, efficiency, copy number control, and for reliable gene response control.

Targeted modification can be achieved either through a nuclease-independent approach, or through a nuclease-dependent approach. In the nuclease-independent targeted editing approach, homologous recombination is guided by homologous sequences flanking an exogenous polynucleotide to be inserted, through the enzymatic machinery of the host cell.

Alternatively, targeted modification could be achieved with higher frequency through specific introduction of double strand breaks (DSBs) by specific rare-cutting endonucleases. Such nuclease-dependent targeted editing utilizes DNA repair mechanisms including non-homologous end joining (NHEJ), which occurs in response to DSBs. Without a donor vector containing exogenous genetic material, the NHEJ often leads to random insertions or deletions (in/dels) of a small number of endogenous nucleotides. In comparison, when a donor vector containing exogenous genetic material flanked by a pair of homology arms is present, the exogenous genetic material can be introduced into the genome during homology directed repair (HDR) by homologous recombination, resulting in a “targeted integration.”

In some embodiments, non-limiting examples of targeted nucleases include naturally occurring and recombinant nucleases; CRISPR related nucleases from families including cas, cpf, cse, csy, csn, csd, cst, csh, csa, csm, and cmr; restriction endonucleases; meganucleases; homing endonucleases, and the like.

In an exemplary embodiment, the CRISPR/Cas9 gene editing technology is used for producing the genetically engineered pluripotent stem cells. Typically, CRISPR/Cas9 requires two major components: (1) a Cas9 endonuclease and (2) the crRNA-tracrRNA complex. When co-expressed, the two components form a complex that is recruited to a target DNA sequence comprising PAM and a seeding region near PAM. The crRNA and tracrRNA can be combined to form a chimeric guide RNA (gRNA) to guide Cas9 to target selected sequences. These two components can then be delivered to mammalian cells via transfection or transduction. Any known CRISPR/Cas9 methods can be used in the methods disclosed herein. See also Examples below.

Besides the CRISPR method disclosed herein, additional gene editing methods as known in the art can also be used in making the genetically engineered T cells disclosed herein. Some examples include gene editing approaching involve zinc finger nuclease (ZFN), transcription activator-like effector nucleases (TALEN), restriction endonucleases, meganucleases homing endonucleases, and the like.

ZFNs are targeted nucleases comprising a nuclease fused to a zinc finger DNA binding domain (ZFBD), which is a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include, but not limited to, C2H2 zinc fingers, C3H zinc fingers, and C4 zinc fingers. A designed zinc finger domain is a domain not occurring in nature whose design/composition results principally from rational criteria, e.g., application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496. A selected zinc finger domain is a domain not found in nature whose production results primarily from an empirical process such as phage display, interaction trap or hybrid selection. ZFNs are described in greater detail in U.S. Pat. Nos. 7,888,121 and 7,972,854. The most recognized example of a ZFN is a fusion of the FokI nuclease with a zinc finger DNA binding domain.

A TALEN is a targeted nuclease comprising a nuclease fused to a TAL effector DNA binding domain. A “transcription activator-like effector DNA binding domain”, “TAL effector DNA binding domain”, or “TALE DNA binding domain” is a polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-diresidues (RVD). TALENs are described in greater detail in U.S. Patent Application No. 2011/0145940, which is incorporated by reference in its entirety. The most recognized example of a TALEN in the art is a fusion polypeptide of the FokI nuclease to a TAL effector DNA binding domain.

Additional examples of targeted nucleases suitable for use as provided herein include, but are not limited to, Bxb1, phiC31, R4, PhiBT1, and WB/SPBc/TP901-1, whether used individually or in combination.

Any of the gene editing nucleases disclosed herein may be delivered using a vector system, including, but not limited to, plasmid vectors, DNA minicircles, retroviral vectors, lentiviral vectors, adenovirus vectors, poxvirus vectors; herpesvirus vectors and adeno-associated virus vectors, and combinations thereof.

Conventional viral and non-viral based gene transfer methods can be used to introduce nucleic acids encoding nucleases and donor templates in cells (e.g., T cells). Non-viral vector delivery systems include DNA plasmids, DNA minicircles, naked nucleic acid, and nucleic acid complexed with a delivery vehicle such as a liposome or poloxamer. Viral vector delivery systems include DNA and RNA viruses, which have either episomal or integrated genomes after delivery to the cell.

Methods of non-viral delivery of nucleic acids include electroporation, lipofection, microinjection, biolistics, virosomes, liposomes, immunoliposomes, polycation or lipid:nucleic acid conjugates, naked DNA, naked RNA, capped RNA, artificial virions, and agent-enhanced uptake of DNA. Sonoporation using, e.g., the Sonitron 2000 system (Rich-Mar) can also be used for delivery of nucleic acids.

### Endoderm Differentiation

The in vitro culturing system disclosed herein may involve a step of endoderm differentiation to differentiate any of the PSCs disclosed herein to definitive endoderm.

Suitable conditions for endoderm differentiation are known in the art (see, e.g., Hay 2008, Brolen 2010 and Duan 2010, and WO 2009/013254 A1) and/or disclosed in Examples below. As used herein “definitive endoderm (DE)” and “definitive endoderm cells (DE cells)” refers to cells exhibiting protein and/or gene expression as well as morphology typical to cells of the definitive endoderm or a composition comprising a significant number of cells resembling the cells of the definitive endoderm. The definitive endoderm is the germ cell layer which gives rise to cells of the intestine, pancreas, liver and lung. DE cells may generally be characterized, and thus identified, by a positive gene and protein expression of the endodermal markers FOXA2, CXCR4, HHEX, SOX17, GATA4 and GATA6. The two markers SOX17 and CXCR4 are specific for DE and not detected in hPSC, hepatic progenitor cells or hepatocytes. Lastly, DE cells do not exhibit gene and protein expression of the undifferentiated cell markers Oct4, SSEA-3, SSEA-4, TRA-1-60, TRA-1-81, but can show low Nanog expression.

Generally, in order to obtain DE cells, PSCs such as hPSC cells can be cultured in an endoderm differentiation medium comprising activin, such as activin A or B. The endoderm differentiation medium may further include a histone deacetylase (HDAC) inhibitor, such as Sodium Butyrate (NaB), Phenylbutyrate (PB), valproate, trichostatin A, Entinostat or Panobinstat. The endoderm differentiation medium may optionally further comprise one or more growth factors, such as FGF1, FGF2 and FGF4, and/or serum, such as FBS or FCS or a serum replacement such as B27+insulin. The endoderm differentiation medium may comprise a GSK3-inhibitor, such as, e.g., CHIR99021, or an activator of Wnt signaling, such as Wnt3A. The endoderm differentiation medium may further include a Rho-associated protein kinase (ROCK) inhibitor. Non-limiting examples of Rho-associated protein kinase (ROCK) inhibitors include, but are not limited to, Y27632, HA-100, H-1152, (+)-trans-4-(1-aminoethyl)-1-(pyridin-4-ylaminocarbonyl) cyclohexane dihydro-chloride monohydrate (described in WO0007835 & WO00057913), imidazopyridine derivatives (described in U.S. Pat. No. 7,348,339, which is incorporated by reference in its entirety), substituted pyrimidine and pyridine derivatives (described in U.S. Pat. No. 6,943,172, which is incorporated by reference in its entirety) and substituted isoquinoline-sulfonyl compounds (described in EP00187371), or GSK429286A, or Thiazovivin, or an analog or derivative thereof.

The concentration of activin is usually in the range of about 50 to about 200 ng/ml, such as about 80 to about 120 ng/ml. Activin may, for example, be present in the endoderm differentiation medium at a concentration of about 90 ng/ml or about 100 ng/ml. As used herein, the term “Activin” is intended to mean a TGF-beta family member that exhibits a wide range of biological activities including regulation of cellular proliferation and differentiation such as “Activin A” or “Activin B”. Activin belongs to the common TGF-beta superfamily of ligands.

The concentration of the HDAC inhibitor is usually in the range of about 0.1 to about 1 mM. The HDAC inhibitor may, for example, be present in the endoderm differentiation medium at a concentration of about 0.4 mM or about 0.5 mM. In one aspect, the HDAC inhibitor is removed from the endoderm differentiation medium after about 3 days. In another aspect, the HDAC inhibitor is added on day 2 and removed on day 5 of culturing PSCs in an endoderm differentiation medium. As used herein HDAC inhibitors refers to Histone deacetylase inhibitors, such as Sodium Butyrate (“NaB”), Phenyl Butyrate (“PB”), Trichostatin A and Valproic Acid (“VA”).

The concentration of serum, if present, is usually in the range of about 0.1 to about 2% v/v, such as about 0.1 to about 0.5%, about 0.2 to about 1.5% v/v, about 0.2 to about 1% v/v, about 0.5 to 1% v/v or about 0.5 to about 1.5% v/v. Serum may, for example, if present, in the endoderm differentiation medium may be at a concentration of about 0.2% v/v, about 0.5% v/v or about 1% v/v. In one aspect, the endoderm differentiation medium omits serum and instead comprises a suitable serum replacement such as B27+insulin.

The concentration of the activator of Wnt signaling is usually in the range of about 0.05 to about 90 ng/ml, such as about 50 ng/ml. As used herein, “activator of Wnt signaling” refers to a compound which activates Wnt signaling. The concentration of the GSK3 inhibitor, if present, is usually in the range of about 0.1 to about 10 μM, such as about 0.05 to about 5 μM. The concentration of the ROCK inhibitor, if present, is typically in the range of 1 μM to about 20 μM, such as 10 μM.

The culture medium forming the basis for the endoderm differentiation medium may be any culture medium suitable for culturing PS cells such as RPMI 1640 or advanced medium, Dulbecco's Modified Eagle Medium (DMEM), HCM medium, HBM medium or Williams E based medium. Thus, the differentiation medium may be RPMI 1640 or advanced medium comprising or supplemented with the above-mentioned components. Alternatively, the differentiation medium may be DMEM comprising or supplemented with the above-mentioned components. The endoderm differentiation medium may thus also be HCM medium comprising or supplemented with the above-mentioned components. The endoderm differentiation medium may thus also be HBM medium comprising or supplemented with the above-mentioned components. The endoderm differentiation medium may thus also be Williams E based medium comprising or supplemented with the above-mentioned components. In one embodiment, the endoderm differentiation medium comprises RPMI1640 containing, in a range of about 1-3%, B27 serum replacement (ThermoFisher).

In some embodiments, the endoderm differentiation medium comprises, consists essentially of, or consists of, an activin, an inhibitor of class I histone deacetylase and an activator of Wnt signaling pathway or GSK3 inhibitor. In other embodiments, the endoderm differentiation medium comprises, consists essentially of, or consists of, an activin, an activator of Wnt signaling pathway or GSK3 inhibitor and a ROCK inhibitor. In another embodiment, the endoderm differentiation medium comprises, consists essentially of, or consists of 1 mM sodium butyrate, Wnt3a 50 ng/ml and Activin A 100 ng/ml, wherein when ‘consisting of’ the medium includes RPMI and a suitable serum replacement (e.g., B27+insulin). In yet another embodiment, the endoderm differentiation medium comprises, consists essentially of, or consists of, Wnt3a 50 ng/ml, Activin A 100 ng/ml, 10 μM Y 27632, wherein when ‘consisting of’ the medium includes RPMI and a suitable serum replacement (e.g., B27+insulin). In still yet another embodiment, the endoderm differentiation medium comprises, consists essentially of, or consists of, 3 μM CHIR99021, 100 ng/mL Activin A, 1 mM sodium butyrate, wherein when ‘consisting of’ the medium includes RPMI and a suitable serum replacement (e.g., B27+insulin). In another embodiment, the endoderm differentiation medium comprises, consists essentially of, or consists of, 3 μM CHIR99021, 100 ng/ml Activin A, 10 μM Y 27632, wherein when ‘consisting of’ the medium includes RPMI and a suitable serum replacement (e.g., B27+insulin).

The PS cells are normally cultured for up to 6 days in suitable endoderm differentiation medium in order to obtain hepatic progenitor cells. For example, the PS cells may be cultured in suitable differentiation medium for about 4 to about 14 days, such as for about 5 to 8 days. In some embodiments, the PS cells are cultured in a cell culture vessel coated with at least one extracellular matrix protein (e.g., laminin or Matrigel) during contact with the endoderm differentiation medium. In some embodiments, the PS cells are dissociated after about 5 days and placed on a permeable membrane, optionally coated with at least one extracellular matrix protein, in a cell culture vessel with an upper and lower chamber separated by the permeable membrane. The PS cells are then contacted with endoderm differentiation medium for the remaining time to induce DE cells, such as about 1-2 days, The PS cells may be dissociated and collected in suspension (e.g., through contact with TrypLE) and then placed in the cell culture vessel having an upper chamber and a lower chamber separated by a permeable membrane. Suitable cell culture vessels are not particularly limited and can include any vessel or insert added thereto where the upper and lower chambers are separated by a permeable membrane. Suitable examples of permeable membranes include but are not limited to polycarbonate, polyester (PET), and collagen-coated polytetrafluoroethylene (PTFE).

In some examples, the method disclosed herein may be performed by culturing the population of pluripotent stem cells in the endoderm differentiation medium for about 5-8 days. In one specific example, endoderm differentiation can be performed by (a) culturing the population of pluripotent stem cells in a first endoderm differentiation medium for one day, wherein the first endoderm differentiation medium comprises an activin, insulin, the activator of Wnt signaling pathway, and the ROCK inhibitor; (b) culturing the population of pluripotent stem cells in a second endoderm differentiation medium following step (a) for one day, wherein the second endoderm differentiation medium comprises an activin, insulin, the activator of Wnt signaling pathway, and the inhibitor of class I histone deacetylase; (c) culturing the population of pluripotent stem cells in a third endoderm differentiation medium following step (c) for two days, wherein the third endoderm differentiation medium comprises an activin, insulin, the GSK3 inhibitor, and the inhibitor of class I histone deacetylase; (d) culturing the population of pluripotent stem cells in a fourth endoderm differentiation medium following step (c) for one day, wherein the fourth endoderm differentiation medium comprises an activin, insulin, the GSK3 inhibitor, and the ROCK inhibitor; and (e) culturing the population of pluripotent stem cells in a fifth endoderm differentiation medium following step (d) for one day, wherein the fifth endoderm differentiation medium comprises an activin, insulin, and the GSK3 inhibitor. In some instances, after step (c) and prior to step (d), the population of pluripotent stem cells can be placed on a permeable membrane.

### Hepatic Specification

Following the endoderm differentiation step, the obtained DE cells can be further cultured in a hepatic specification medium to obtain hepatic progenitor cells. As used herein, “hepatic progenitors” or “hepatic progenitor cells” refers to cells which have entered the hepatic cell path and give rise to hepatocyte. “Hepatic progenitors” are thus distinguished from “endodermal cells” in that they have lost the potential to develop into cells of the intestine, pancreas and lung. “Hepatic progenitors” may generally be characterized, and thus identified, by a positive gene and protein expression of the early hepatic markers EpCAM, c-Met (HGF-receptor), AFP, CK19, HNF6, C/EBPa and β. They do not exhibit gene and protein expression of the DE-markers CXCR4 and SOX17. Lastly, “hepatic progenitors” do not exhibit gene and protein expression of the undifferentiated cell markers Oct4, SSEA-3, SSEA-4, TRA-1-60 and TRA-1-81 nor the mature hepatic markers CYP1A2, CYP2C9, CYP19, CYP3A4, CYP2B6 and PXR.

In general, in order to obtain hepatic progenitor cells, DE cells are cultured in a hepatic differentiation medium comprising one or more growth factors, such as a fibroblast growth factor (FGF) (e.g., FGF1, FGF2 and FGF4), and one or more bone morphogenic proteins (BMP), such as BMP2 and BMP4. As used herein, the term “FGF” means fibroblast growth factor, preferably of human and/or recombinant origin, and subtypes belonging thereto are e.g. “bFGF” (means basic fibroblast growth factor, sometimes also referred to as FGF2) and FGF4. “aFGF” means acidic fibroblast growth factor (sometimes also referred to as FGF1). As used herein, the term “BMP” means Bone Morphogenic Protein, preferably of human and/or recombinant origin, and subtypes belonging thereto are e.g. BMP4 and BMP2. The concentration of the one or more growth factors may vary depending on the particular compound used. The concentration of FGF2, for example, is usually in the range of about 2 to about 50 ng/ml, such as about 2 to about 20 ng/ml. FGF2 may, for example, be present in the specification medium at a concentration of 9 or 10 ng/ml. The concentration of FGF1, for example, is usually in the range of about 50 to about 200 ng/ml, such as about 80 to about 120 ng/ml. FGF1 may, for example, be present in the specification medium at a concentration of about 100 ng/ml. The concentration of FGF4, for example, is usually in the range of about 20 to about 40 ng/ml. FGF4 may, for example, be present in the specification medium at a concentration of about 30 ng/ml. The concentration of the one or more BMPs, is usually in the range of about 50 to about 300 ng/ml, such as about 50 to about 250 ng/ml, about 100 to about 250 ng/ml, about 150 to about 250 ng/ml, about 50 to about 200 ng/ml, about 100 to about 200 ng/ml or about 150 to about 200 ng/ml. The concentration of BMP2, for example, is usually in the range of about 2 to about 50 ng/ml, such as about 10 to about 30 ng/ml. BMP2 may, for example, be present in the hepatic specification medium at a concentration of about 20 ng/ml.

The culture medium forming the basis for the hepatic specification medium may be any culture medium suitable for culturing human endodermal cells such as RPMI 1640 or advanced medium, Dulbecco's Modified Eagle Medium (DMEM), HCM medium, HBM medium or Williams E based medium. Thus, the hepatic specification medium may be RPMI 1640 or advanced medium comprising or supplemented with the above-mentioned components. Alternatively, the hepatic specification medium may be DMEM comprising or supplemented with the above-mentioned components. The hepatic specification medium may thus also be HCM medium comprising or supplemented with the above-mentioned components. The hepatic specification medium may thus also be HBM medium comprising or supplemented with the above-mentioned components. The hepatic specification medium may thus also be Williams E based medium comprising or supplemented with the above-mentioned components. In some embodiments, the DE cells are cultured in a cell culture vessel coated with at least one extracellular matrix protein (e.g., laminin) during contact with the hepatic specification medium.

In other embodiments, the hepatic specification medium comprises, consists essentially of, or consists of, bFGF and BMP4. In another embodiment, the endoderm differentiation medium comprises, consists essentially of, or consists of 50 ng/ml bFGF and 20 ng/ml BMP4, wherein when ‘consisting of’ the medium includes RPMI and a suitable serum replacement (e.g., B27+insulin).

For specification into hepatic progenitor cells, DE cells are normally cultured for up to 3 days in differentiation medium as described above. The DE cells may, for example, be cultured in differentiation medium for about 2 to about 4 days. In some embodiments, the DE cells are maintained in the cell culture vessel comprising an upper and lower chamber separated by a permeable membrane, optionally coated with at least one extracellular matrix protein, during specification to hepatic progenitor cells, wherein the DE cells are in contact with the permeable membrane.

### Hepatocyte Maturation

The hepatocyte progenitor cells obtained from the hepatocyte specification step may be further cultured in a hepatic maturation medium to obtain the hepatocyte-like cells. As used herein, “hepatocyte” or “hepatocyte-like cells” refers to fully differentiated hepatic cells. “Hepatocytes” or “hepatocytes-like cells” may generally be described, and thus identified, by a positive gene and protein expression of the mature hepatic markers CYP1A2, CYP3A4, CYP2C9, CYP2C19, CYP2B6, GSTA1-1, OATP-2, NTCP, Albumin, PXR, CAR, and HNF4a (isoforms 1+2) among others. Further, “hepatocytes” or “hepatocyte-like cells do not exhibit gene and protein expression of the undifferentiated cell markers Oct4, SSEA-3, SSEA-4, TRA-1-60 and TRA-1-81. Compared to DE cells, “hepatocytes” or “hepatocyte-like cells do not exhibit gene and protein expression of the DE cell markers SOX17 and CXCR4. Compared to “hepatic progenitors”, “hepatocytes” or “hepatocyte-like cells do not exhibit gene and protein expression of the hepatic progenitor markers Cytokeratin 19 and AFP. As meant herein, a gene or protein shall be interpreted as being “expressed”, if in an experiment measuring the expression level of said gene or protein, the determined expression level is higher than three times the standard deviation of the determination, wherein the expression level and the standard deviation are determined in 10 separate determinations of the expression level. The determination of the expression level in the 10 separate determinations is preferably corrected for background-signal. Moreover, the ‘hepatocyte-like cells’ is meant to include cells which have similar functionalities as primary hepatocytes, and in particular show phenotypical features of functional hepatocytes when exposed to bile acids. Said phenotypical features may include expression and polarization of bile acid transport proteins, uptake, transport, synthesis and/or excretion of bile acids at a level similar to primary hepatocytes. In particular, in the context of the present invention, hepatocyte-like cells are meant to include human embryonic stem cells differentiated into hepatocyte-like cells, human induced pluripotent stem cells differentiated into hepatocyte-like cells, or primary fibroblast transdifferentiated into hepatocyte-like cells.

In general, in order to obtain hepatocyte-like cells, hepatic progenitor cells are cultured in a hepatocyte maturation medium comprising one or more of a hepatocyte growth factor (HGF), one or more differentiation inducer (e.g., such as dimethylsulfoxide (DMSO), dexamethazone (DexM), omeprazole, Oncostatin M (OSM), rifampicin, desoxyphenobarbital, ethanol or isoniazide), transferrin, hydrocortisone and insulin, where the hepatocyte maturation medium preferably omits human epidermal growth factor (EGF). As used herein, the term “HGF” means Hepatocyte Growth Factor, preferably of human and/or recombinant origin. As used herein, the term “EGF” means Epidermal Growth Factor, preferably or human and/or recombinant origin.

The concentration of HGF, is usually in the range of about 5 to about 30 ng/ml. HGF may, for example, be present in the differentiation medium at a concentration of about 20 ng/ml. The concentration of DMSO, for example, is usually in the range of about 0.1 to about 2% v/v, such as about 0.1 to about 1.5% v/v, about 0.1 to about 1% v/v, about 0.25 to about 1% v/v, about 0.25 to about 0.75% v/v, about 0.5 to about 1.5% v/v, or about 0.5 to about 1% v/v. The concentration of OSM, for example, is usually in the range of about 1 to about 20 ng/ml, such as about 1 to about 15 ng/ml, about 5 to about 15 ng/ml, or about 7.5 to about 12.5 ng/ml. The concentration of DexM, for example, is usually in the range of about 0.05 to about 1 μM, such as about 0.05 to about 0.5 μM, about 0.05 to about 0.2 μM, about 0.05 to about 0.1 μM or about 0.1 to about 0.5 μM.

The hepatocyte maturation medium may further comprise serum, such as FBS or FCS. The concentration of serum, if present, is usually in the range of about 0.1 to about 5% v/v, such as about 0.1 to about 0.5%, 0.2 to 3% v/v, about 0.5 to about 2.5% v/v, about 0.5 to 1% v/v or about 1 to about 2.5% v/v. In some embodiments, the hepatocyte maturation medium further comprises one or more of BSA-fatty acid free (BSA-FAF), ascorbic acid, and GA-1000.

The culture medium forming the basis for the hepatocyte maturation medium may be any culture medium suitable for culturing human endodermal cells such as RPMI 1640 or advanced medium, Dulbecco's Modified Eagle Medium (DMEM), HCM medium, HBM medium or Williams E based medium. Thus, the hepatocyte maturation medium may be RPMI 1640 or advanced medium comprising or supplemented with the above-mentioned components. Alternatively, the hepatocyte maturation medium may be DMEM comprising or supplemented with the above-mentioned components. The hepatocyte maturation medium may thus also be HCM medium comprising or supplemented with the above-mentioned components. The hepatocyte maturation medium may thus also be HBM medium comprising or supplemented with the above-mentioned components. The hepatocyte maturation medium may thus also be Williams E based medium comprising or supplemented with the above-mentioned components.

In some embodiments, the hepatocyte maturation step preferably omits co-culture of the hepatic progenitor cells with any other cell type. In a specific aspect, the hepatocyte maturation step omits co-culture human umbilical vein endothelial cells (HUVEC) and/or mesenchymal stem cells (MSC) to produce a population of hepatocyte-like cells.

For differentiation into hepatocyte-like cells, hepatic progenitor cells are normally cultured for up to 14 days (e.g., up to 12 days) in the hepatocyte maturation medium as described above. The hepatic progenitor cells may, for example, be cultured in differentiation medium for about 12 to about 16 days (e.g., for about 12-14 days). In some embodiments, the hepatic progenitor cells are maintained in the cell culture vessel comprising an upper and lower chamber separated by a permeable membrane, optionally coated with at least one extracellular matrix protein, during maturation to hepatocyte-like cells, wherein the hepatic progenitor cells are in contact with the permeable membrane.

### In Vitro Cell Culturing Systems and Uses Thereof

Any of the hepatocyte-like cells produced by the methods of various aspects described herein can be used in different applications where hepatocytes are required. Such hepatocyte-like cells are also within the scope of the present disclosure. For example, in some embodiments, the hepatocyte-like cells for use in the in vitro system described herein may have a normal BSEP gene. In some embodiments, the hepatocyte-like cells are unmodified hepatocyte-like cells (e.g., hepatocyte like-cells produced from wild-type PS cells) and may show a functional apico-basolateral polarity, transport of bile acids and/or de novo synthesis of bile acids. In some embodiments, the bile acid transport ability is important for the allo-antibody detection methods.

In some aspect, provided herein is an in vitro cell culture system, which comprises a two-chamber cell culture vessel. In some embodiments, the cell culture vessel comprises:
a cell culture vessel comprising an upper chamber and a lower chamber; wherein both the upper chamber and the lower chamber comprise a medium for culturing hepatocytes;
a permeable membrane separating the upper chamber and the lower chamber; and
a layer of hepatocyte-like cells grown on the permeable membrane.

In one aspect, the in vitro cell culture system comprises hepatocyte-like cells differentiated from a population of pluripotent stem cells having a modified ABCB11 gene. In some embodiments, the permeable membrane is optionally coated with at least one extracellular matrix protein, in a cell culture vessel with an upper and lower chamber separated by the permeable membrane. As noted above, suitable cell culture vessels are not particularly limited and can include any multi-well vessel comprising a permeable membrane as a barrier between wells or an insert may be added to a single well vessel thereby producing an upper and lower chamber separated by the permeable membrane. Suitable examples of permeable membranes include but are not limited to polycarbonate, polyester (PET), and collagen-coated polytetrafluoroethylene (PTFE).

The method comprises (i) providing an in vitro cell culture system as disclosed herein (ii) adding a bile acid (e.g., taurocholic acid (TCA) to the lower chamber, (iii) culturing the hepatocyte-like cells in the presence of a candidate agent; (iv) measuring the concentration of the bile acid in the upper chamber and/or in the lower chamber. In some embodiments, the candidate agent is identified the candidate agent as an agent for treating a cholestatic liver disease if the candidate agent changes the bile acid concentration determined in step (iv) as compared with the in vitro cell culture system in the absence of the candidate agent.

The candidate agents can be selected from the group consisting of proteins, peptides, nucleic acids (e.g., but not limited to, siRNA, anti-miRs, antisense oligonucleotides, and ribozymes), small molecules, nutrients (lipid precursors), and a combination of two or more thereof.

In some embodiments, effects of the candidate agents on the hepatocyte-like cells of the disclosure can be determined by measuring response of the cells and comparing the measured response with hepatocyte-like cells that are not contacted with the candidate agents. Various methods to measure cell response are known in the art, including, but not limited to, cell labeling, immunostaining, optical or microscopic imaging {e.g., immunofluorescence microscopy and/or scanning electron microscopy), spectroscopy, gene expression analysis, cytokine/chemokine secretion analysis, metabolite analysis, polymerase chain reaction (PCR), immunoassays, ELISA, gene arrays, spectroscopy, immunostaining, electrochemical detection, polynucleotide detection, fluorescence anisotropy, fluorescence resonance energy transfer, electron transfer, enzyme assay, magnetism, electrical conductivity (e.g., trans-epithelial electrical resistance (TEER)), isoelectric focusing, chromatography, immunoprecipitation, immunoseparation, aptamer binding, filtration, electrophoresis, use of a CCD camera, mass spectroscopy, or any combination thereof. Detection, such as cell detection, can be carried out using light microscopy with phase contrast imaging and/or fluorescence microscopy based on the characteristic size, shape and refractile characteristics of specific cell types.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Intracellular accumulation of conjugated bile acids in BSEP deficient hepatocytes has been proposed since conjugated bile acids are not excreted in the bile and are found in the liver in high concentration. However, direct evidence of intracellular accumulation of bile acids in human hepatocytes is lacking. In this report, new insights into the mechanism of cellular regulation of intracellular bile acids are provided. By using a newly established in vitro system of human hepatocytes, which recapitulates the expression pattern of truncated BSEP, it was found that hepatocytes with BSEP deficiency in part use basolateral transporters, MRP4, to export conjugated bile acids in order to prevent their intracellular accumulation.

Hepato-enteric bile acid circulation reaches homeostasis by the interaction between transcellular bile acid transport and de novo synthesis mediated by intracellular bile acids in hepatocytes. i-Hep in culture system described herein synthesized de novo bile acids at the last stage of the hepatic differentiation under the regulation of HGF, consistent with previous reports of spontaneous bile acid synthesis and secretion by cultured hepatocytes. Ellis et al., Methods Mol Biology Clifton N J 640:417-430 (2010); Liu et al., Toxicol Sci 141:538-546 (2014); and Einarsson et al., World J Gastroentero 6:522-525 (2000). It has been demonstrated that human hepatocytes develop regulatory mechanisms to control the concentration of intracellular conjugated bile acids when BSEP is genomically deficient. The BSEP deficient hepatocytes export endogenous conjugated bile acids via the basolateral membrane as they mature. In patients with PFIC2, since sinusoidal bile acids do not flow into the hepato-enteric circulation, they remain in the systemic circulation, leading to jaundice and cholestasis. The mechanisms regulating bile acids accumulating in the systemic circulation and de novo bile acid synthesis have not been defined previously.

It has been demonstrated BSEP deficient hepatocytes are able to down-regulate de novo bile acid synthesis via the uptake and export of bile acids on the basolateral domain, while preventing accumulation of intracellular bile acids. This suggests that BSEP deficient hepatocytes can achieve homeostasis of bile acids concentration of the systemic circulation.

The analysis of ultrastructure showed structural disturbance of the basolateral membrane in BSEP^{R1090X} i-Hep. Previous studies showed that increased concentration of bile acids increases lipid fluidity of plasma membrane and disrupt membrane functional domain. Scharschmidt et al., Hepatology 1:137-145 (1981). It was speculated that constant intracellular-to-basolateral reflux of bile acid may cause abnormally increased concentration of bile acids between the lateral membranes of adjunct cells, thus induce membrane degradation or instability. Given that these changes were found in the liver of patients with PFIC2, they may be important pathophysiological features of BSEP deficiency.

This system allows for directly determination of the cellular and biochemical effect of previously unreported genetic variants and the molecular consequence of missense mutations, often reported as “variant of unknown clinical significance”. As the knowledge of disease-causing variants further accumulates, it would be relied on to predict the clinical course from the genotype and design personalized management strategies at an early stage of the disease. In addition, this system allows for studying whether gene therapy or tissue transplantation is a viable option for subject with known disease. Further, this system will allow for monitoring of a patient who does receive gene therapy or tissue transplantation to ensure the subject is not rejecting the therapy or transplanted tissue by periodically taking serum samples and studying whether the cells transport bile acid from the lower chamber to the upper chamber.

In summary, these findings reveal novel mechanisms that underlie the pathophysiology of BSEP deficiency and provide targets for therapeutic intervention in patients with PFIC2. Further, the described system allows for understanding of how a subject may react to a certain therapy or how a subject is reacting to a certain therapy by studying whether antibodies are present in a serum sample using this system.

### Use of the System to Study the Presence or Absence of Antibodies in a Sample

After the cells have been cultured by the above methods and components, additional steps are taken to determine the presence or absence of an allo-antibody in a serum sample.

The method for detecting the presence of an allo-antibody against bile salt export pump (BSEP) protein in a subject, comprises the steps of:
(a) providing a cell culture vessel comprising (i) an upper chamber and a lower chamber, wherein both the upper chamber and the lower chamber comprise a culture medium for culturing hepatocytes, (ii) a permeable membrane separating the upper chamber and the lower chamber, and (iii) a layer of hepatocyte-like cells grown on the permeable membrane, wherein the hepatocyte-like cells are differentiated from a population of pluripotent stem cells having a modified ABCB11 gene;
(b) adding taurocholic acid (TCA) to the lower chamber culture medium;
(c) adding a serum sample from the subject to the upper chamber culture medium;
(d) after a period of time, collecting culture medium from each of the upper chamber and the lower chamber; and
(e) determining the concentration of TCA in the culture medium from each of the upper chamber and the lower chamber;
   wherein the lack of TCA in the culture medium of the upper chamber indicates that the sample contains allo-antibody against BSEP.

That is, the same culture system and vessel can now be used as a bile acid transport assay. In this regard, bile acid can be used to determine whether an allo-antibody is present in a subjects sample.

Taurocholic acid (TCA) is a known bile acid involved in the emulsification of fats. The sodium salt of this acid can be found in the bile of mammals. TCA is a conjugate of cholic acid with taurine. TCA is added to the lower chamber culture medium. TCA can be added in an amount of about 0.01 mM to about 1 mM, preferably about 0.05 mM to about 0.5 mM. This assay studies the amount of TCA that is transported from this lower chamber into the upper chamber through the transwell permeable membrane, optionally coated with at least one extracellular matrix protein, through the BSEP function.

Further, a serum sample from the subject is added to the upper chamber culture medium. The serum sample can be taken prior to therapy at the time the cells are cultured, during therapy, and after therapy. In some embodiments, the serum sample is maintained in the cell culture in the upper chamber of a vessel comprising an upper and lower chamber separated by the permeable membrane, wherein the serum is in in contact with the permeable membrane and the cultured hepatocyte-like cells.

This assay can be used as a way for medical professional to monitor the progress of the disease and treatment by taking serum samples at various points before, during, and after treatment with a gene therapy or transplantation. The samples may be taken from the subject weekly, bi-weekly or monthly. In some embodiments, the samples are taken weekly for two months after treatment. The subject can then be monitored monthly after the initial two month. The number and frequency for obtaining a sample will vary depending on the therapy administered and the ongoing real-time results gathered. In some embodiments, the samples will be obtained with greater frequency. In other embodiments, the samples will be obtained with lesser frequency. The amount and frequency for obtaining the sample will be determined by the medical professionals monitoring the subject progress.

After the addition of the serum sample to the upper chamber and TCA to the lower chamber and after a period of time, the culture medium is collected from each of the upper chamber and the lower chamber. The period of time can be from about 2 hours to about 1 weeks, preferably about 2 hours to about 48 hours after the culture serum sample of the subject has been added, more preferably about 48 hours. Further, the cultural medium is collected via conventional methods of collection and stored at −20° C.

After collecting the culture medium of the upper and lower chambers, the concentration of TCA in the culture medium from each chamber is determined. The concentration is TCA is determined by known methods, such as a general titration method. A titration method includes adding a known concentration of base to the acid solution until the endpoint is reached. This method allows one to calculate the moles of acid based on the volume of base used and the balanced chemical reaction involved. The percentage of TCA in the upper chamber can be calculated by dividing the concentration of TCA in the upper chamber by the sum of the concentration of TCA in the upper chamber and the concentration of TCA in the lower chamber.

In some embodiments, the bile acid concentration is determined by a commercially available kit called The Diazyme Total Bile Acids (TBA) Assay, which can be found on the Diazyme website. Diazyme described that assay as “a liquid stable system, ready to use for both manual methods and is adaptable for many automated chemistry analyzers. The assay has excellent sensitivity with a linear range from 0-180 μM.” Further, Diazyme states, “In the presence of Thio-NAD, the enzyme 3-α-hydroxysteroid dehydrogenase (3-α-HSD) converts bile acids to 3-keto steroids and Thio-NADH. The reaction is reversible and 3-α-HSD can convert 3-keto steroids and Thio-NADH to bile acids and Thio-NAD. In the presence of excess NADH, the enzyme cycling occurs efficiently and the rate of formation of Thio-NADH is determined by measuring specific change of absorbance at 405 nm.”

The concentration of TCA in the upper chamber can be indicative of the presence or absence of an allo-antibody. Specifically, the lack of TCA in the culture medium of the upper chamber indicates that the sample contains an allo-antibody against BSEP. Specifically, the lack of TCA in the culture medium of the upper chamber means that no TCA was transported from the lower chamber to the upper chamber and indicates that the serum sample contains anti-BSEP allo-antibody. The presence of anti-BSEP allo-antibody indicates the subject may likely exhibit gene therapy/graft rejection, and in the case of a PFIC2 subject, recurrence of BSEP deficiency.

Conversely, TCA in the upper chamber culture medium indicates that the serum sample does not contain anti-BSEP antibodies and the hepatocytes transported the TCA from the lower chamber to the upper chamber. The presence of TCA in the upper chamber indicates the subject is unlikely to exhibit gene therapy/graft rejection or recurrence of BSEP deficiency. Further, during monitoring, the presence of TCA in the upper chamber can indicate that the subject is tolerating the therapy well and is not rejecting the gene therapy or graft tissue.

In some embodiments, the indication of allo-antibodies based on the presence of TCA may be determined on a gradient. That is, when the concentration of TCA is determined to be at about 25% of the TCA added to the lower chamber or less, it may be determined that the allo-antibodies are present. Further, when the concentration of TCA is about 26% to about 75% of the added TCA, it may be indeterminate that the allo-antibodies are present or present in an amount that indicates possible rejection of gene therapy or transplanted tissue. In such a case, further monitoring or study takes place over time to determine whether a change in the TCA concentration of further samples changes. Still further, when the concentration of TCA is above about 76% or more, it can be determined that an allo-antibody is not present indicating that the subject is unlikely to exhibit gene therapy/graft rejection or recurrence of BSEP deficiency.

In another embodiment, the two chamber system may be prepared in a different manner. That is, a method of predicting autoimmunity post gene therapy for PFIC2 comprises (a) providing a cell culture vessel comprising (i) an upper chamber and a lower chamber, wherein both the upper chamber and the lower chamber comprise a culture medium for culturing hepatocytes, (ii) a permeable membrane, optionally coated with at least one extracellular matrix protein, separating the upper chamber and the lower chamber, and (iii) a layer of hepatocyte-like cells grown on the permeable membrane, wherein the hepatocyte-like cells are differentiated from a population of pluripotent stem cells having a modified ABCB11 gene; (b) adding taurocholic acid (TCA) to the lower chamber culture medium; (c) adding a serum sample from the subject to the upper chamber culture medium; (d) after a period of time, collecting culture medium from each of the upper chamber and the lower chamber; and (e) determining the concentration of TCA in the culture medium from each of the upper chamber and the lower chamber; wherein the lack of TCA in the culture medium of the upper chamber indicates that the subject providing the sample will exhibit autoimmunity post gene therapy.

As can be seen, this assay and method may critical for determining the most suitable treatment method for a subject with cholestasis or a subject with PFIC2. Further, this assay and method are also crucial tools for monitoring the progress of therapy after administered. This tool is one not presently available for medical professionals treating patients with cholestasis or PFIC2.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

It should also be understood that, unless clearly indicated to the contrary, in any methods claimed herein that include more than one step or act, the order of the steps or acts of the method is not necessarily limited to the order in which the steps or acts of the method are recited.

1. An method for detecting the presence of an allo-antibody against bile salt export pump (BSEP) protein in a subject, comprising:
(a) providing a cell culture vessel comprising (i) an upper chamber and a lower chamber, wherein both the upper chamber and the lower chamber comprise a culture medium for culturing hepatocytes, (ii) a permeable membrane separating the upper chamber and the lower chamber, and (iii) a layer of hepatocyte-like cells grown on the permeable membrane, wherein the hepatocyte-like cells are differentiated from a population of pluripotent stem cells having a modified ABCB11 gene;
(b) adding taurocholic acid (TCA) to the lower chamber culture medium;
(c) adding a serum sample from the subject to the upper chamber culture medium;
(d) after a period of time, collecting culture medium from each of the upper chamber and the lower chamber; and
(e) determining the concentration of TCA in the culture medium from each of the upper chamber and the lower chamber;
wherein the lack of TCA in the culture medium of the upper chamber indicates that the sample contains allo-antibody against BSEP.

2. The method of claim 1, wherein the period of time is about 48 hours.

3. The method of claim 2, further comprising, after step (d), culturing the cells for an additional period of time, and collecting culture medium from each of the upper chamber and the lower chamber, and determining the concentration of TCA in the culture medium from each of the upper chamber and the lower chamber.

4. The method of claim 3, wherein the additional period of time is about 24 hours.

5. The method of claim 1, wherein the concentration of TCA in the lower chamber is about 0.01 mM to about 1 mM.

6. The method of claim 1, wherein when the concentration of TCA is about 25% of the TCA added to the lower chamber or less, it is determined that the allo-antibodies are present; when the concentration of TCA is about 26% to about 75% of the added TCA to the lower chamber, it is indeterminate that the allo-antibodies are present; and when the concentration of TCA is above about 76% or more of the TCA added to the lower chamber, the allo-antibodies are not present.

7. A method of predicting autoimmunity post gene therapy for PFIC2 comprising:
(a) providing a cell culture vessel comprising (i) an upper chamber and a lower chamber, wherein both the upper chamber and the lower chamber comprise a culture medium for culturing hepatocytes, (ii) a permeable membrane separating the upper chamber and the lower chamber, and (iii) a layer of hepatocyte-like cells grown on the permeable membrane, wherein the hepatocyte-like cells are differentiated from a population of pluripotent stem cells having a modified ABCB11 gene;
(b) adding taurocholic acid (TCA) to the lower chamber culture medium;
(c) adding a serum sample from the subject to the upper chamber culture medium;
(d) after a period of time, collecting culture medium from each of the upper chamber and the lower chamber; and
(e) determining the concentration of TCA in the culture medium from each of the upper chamber and the lower chamber;
wherein the lack of TCA in the culture medium of the upper chamber indicates that the subject providing the sample will exhibit autoimmunity post gene therapy.

8. The method of claim 7, wherein the additional period of time is about 24 hours.

9. The method of claim 7, wherein the concentration of TCA in the lower chamber is about 0.01 mM to about 1 mM.

10. The method of claim 7, wherein when the concentration of TCA is about 25% of the TCA added to the lower chamber or less, it is determined that the allo-antibodies are present; when the concentration of TCA is about 26% to about 75% of the added TCA to the lower chamber, it is indeterminate that the allo-antibodies are present; and when the concentration of TCA is above about 76% or more of the TCA added to the lower chamber, the allo-antibodies are not present.
